(19) United States
(12) Patent Application Publication
(10) Pub. No.: US 2023/0320843 A1
(43) Pub. Date: Oct. 12, 2023

(54) Ossicular Prosthesis

(71) Applicant: MED-EL Elektromedizinische Geräte GmbH, Innsbruck (AT)

(72) Inventors: Lukas Mair, Schwaz (AT); Uwe Steinhardt, Hirrlingen (DE); Clemens Kuen, Imst (AT)

(21) Appl. No.: 18/041,098

(22) PCT Filed: May 27, 2021
(86) PCT No.: PCT/EP2021/064147

§ 371 Date: Feb. 9, 2023

(30) Foreign Application Priority Data
Aug. 13, 2020 (EP) 20190807.6

(51) Int. Cl. A61F 2/18

(52) U.S. Cl. CPC A61F 2/18; A61F 2002/183; A61F 2220/0016; A61F 2230/0013; A61F 2250/0007

(57) ABSTRACT

The invention proposes forming an auditory ossicle prosthesis (1) in one piece from sheet metal, like pliers, with two congruent clamps (3), which can be opened and closed with a two-part head element (2), the head parts (4) of which act as pliers handles. The auditory ossicle prosthesis (1) can be clamped onto an auditory ossicle without exerting any force on the auditory ossicle.

### FIELD OF THE INVENTION

The invention relates to an auditory ossicle prosthesis having the features of the preamble of claim 1.

### BACKGROUND

Auditory ossicle prostheses serve as a replacement for one or more auditory ossicles in the human middle ear and, instead of the replaced auditory ossicles, transmit vibrations of the eardrum generated by sound waves to the oval window of the inner ear. The length of an auditory ossicle prosthesis must be individually adapted to a distance to be bridged from the eardrum to the auditory ossicles to be coupled, for example the stapes of the respective patient.

European patent EP 3 512 463 B1 discloses a one-piece ossicular prosthesis which is separated from a piece of sheet metal and bent to form the auditory ossicular prosthesis. The known auditory ossicle prosthesis has a planar head element and two U-shaped clamps protruding from the head element. The head element has a flat, rectangular central part with Ω-shaped brackets arranged mirror-symmetrically on opposite sides of the central part in one plane with the central part, the feet of which merge into the central part in one piece. The head element is intended to rest on an inside of the eardrum. Between the Ω-shaped brackets, the two U-shaped clamps protrude almost at right angles to the head element on the same side. When viewed from the side, the two Ω -shaped terminals are congruent. Their yokes are integrally connected to the central part of the head element via short bars bent almost at right angles to the plane of the head element. Legs of the U-shaped clamps form clamping jaws for a clamping placement on an auditory ossicle. The legs of the U-shaped clamps forming the jaws are zigzag-shaped. A disadvantage of the known auditory ossicle prosthesis is that the zigzag-shaped clamping jaws exert a force on the auditory ossicles when they are placed on the auditory ossicles.

The object of the invention is to specify an auditory ossicle prosthesis of the type explained above, which can be placed more easily on an auditory ossicle.

### SUMMARY OF THE INVENTION

According to the invention, this object is achieved by the features of claim ₁. The auditory ossicle prosthesis according to the invention is designed like pliers. It has a two-part or multi-part head element with at least two head parts and one or more clamps with clamping jaws for a clamping placement on an auditory ossicle, in particular on a rod or rod-shaped section of an auditory ossicle. The head element is intended to be arranged on an inside of an eardrum in such a way that vibrations of the eardrum generated by sound waves are transmitted to the auditory ossicle prosthesis. In this context, an eardrum is understood to mean both an existing, partially existing and an artificially complete or partially reconstructed eardrum, as well as a partially existing and otherwise reconstructed eardrum. The reconstruction includes any form of manufacturing an vibratory membrane, for example with body tissue or an artificially created vibrating membrane to convert sound into mechanical vibrations. Also included is an arrangement or attachment of the head element to an auditory ossicle instead of to the eardrum. Two head parts of the head element can be moved relative to one another, in particular their distance from one another can be increased and/or decreased. The clamping jaws can also be moved relative to one another, in particular they can be pivoted relative to one another, so that their distance from one another can also be increased and/or decreased. One of the head parts is connected to one clamping jaw and another head part is rigidly connected to the other clamping jaw, or at least so rigidly that a movement of the head parts relative to one another, in particular by changing their distance from one another, changes the distance between the clamping jaws in a manner comparable to pliers. In particular, the distance between the jaws increases when the heads are brought closer together and vice versa. Embodiments of the auditory ossicle prosthesis according to the invention are also conceivable in which the distance between the clamping jaws increases when the distance between the head parts of the head element increases. A surgeon can place the clamp of the auditory ossicle prosthesis on an auditory ossicle by gripping the head element of the auditory ossicle prosthesis in a initial position with, for example, forceps which he attaches to the two head parts. The surgeon places the forceps on the head element of the auditory ossicle prosthesis from a side opposite the clamp. By closing the forceps, he presses the two head parts together, which reduces their distance from each other. The ossicular prosthesis compressed in this way at the head parts is in a coupling position in which the clamp opens, i.e. the two clamping jaws move away from each other, so that the surgeon can place the clamp or the clamping jaws of the ossicular prosthesis on the ossicles in a particularly advantageous manner, without exerting any force on the auditory ossicles. This prevents damage to the sensitive auditory ossicles. If the surgeon opens the forceps and slowly releases the ossicle prosthesis, the jaws of the clamp spring together and clamp the ossicle prosthesis to the ossicles. In particular, the clamping jaws spring back elastically into the initial position or in contact with the auditory ossicles when the head parts are released. They rest against the auditory ossicles with a preload and thereby hold the auditory ossicle prosthesis on the auditory ossicles.

The forceps will not disengage from the ossicular prosthesis until there is no force on the forceps arms as a result of counteracting the preload from the auditory ossicular prosthesis, which is exactly the case when the entire preload of the ossicular prosthesis is applied to the jaws. This ensures that the forceps are only released from the ossicular prosthesis when the ossicular prosthesis is securely placed and fixed on the ossicle. This considerably simplifies handling and placement during the surgical intervention, for example, and prevents, among other things, unintentional or premature detachment of the auditory ossicle prosthesis from the forceps, for example before exact positioning, alignment and fixation. In addition, forceps can be used that only allow a small holding force to be exerted on the auditory ossicle prosthesis and thus prevent plastic deformation of the auditory ossicle prosthesis.

An additional advantage of the invention is that the clamp of the auditory ossicle prosthesis can be placed on an auditory ossicle without exerting a force on the auditory ossicle during placement.

One embodiment of the invention provides a deformable bar that connects the clamping jaws at adjustable distances. The bar forms a kind of joint; the distance between the clamping jaws can be changed by deforming the bar. In particular, the bar is bent to change the distance between the clamping jaws. This embodiment of the invention enables a one-piece design of the clamp and/or the ossicular prosthesis.

A distance between the clamping jaws can be adjusted to adapt to a thickness of the auditory ossicles in embodiments of the invention by plastic deformation, in particular of the deformable bar connecting the clamping jaws and/or the clamping jaws. The distance between the clamping jaws is set smaller than the thickness of the auditory ossicles, so that the clamping jaws lie elastically against the auditory ossicles with the preload when the clamp of the auditory ossicle prosthesis is placed on the auditory ossicles.

The clamp can be formed by a slot in a region of the ossicular prosthesis that forms the clamp. Areas on both sides of the slot form the clamping jaws, the distance between which can be changed through the slot. One embodiment of the invention provides a U-shaped clamp, with legs of the “U” forming the jaws.

For a better retention on the auditory ossicles, one embodiment of the invention provides at least two clamps. In particular, the clamps are congruent. However, it is sufficient if the clamps are arranged at a distance from one another in such a way that they can be attached on the same auditory ossicles.

A preferred embodiment provides that edges or surfaces of the clamping jaws facing one another have a corrugation, prongs or teeth. This changes the clear distance between the edges or surfaces of the clamping jaws that face one another, the distance increases and decreases alternately in a longitudinal direction of the clamping jaws, and the clamp of the ossicular prosthesis can thus be placed on, for example, a rod or bar-shaped section of the ossicle, so that the rod or rod-shaped section being located in opposite troughs or in opposite spaces between prongs or teeth of the facing edges or surfaces of the clamping jaws. This improves retention of the ossicular prosthesis in the longitudinal direction of its clamping jaws on the ossicles and the distance between the head element and the ossicles can be adjusted in steps equal to the distance between the corrugation troughs or the spaces between the prongs or teeth. As a result, the auditory ossicle prosthesis according to the invention can be easily adapted to a distance between the eardrum and the auditory ossicles.

An embodiment of the invention provides that the distance between the clamping jaws increases from a free end to a closed end of the clamp. This creates an opening between the jaws at the free end. In the case of wavy or jagged edges or surfaces of the clamping jaws facing one another, the distances between opposing corrugation crests and/or corrugation troughs or opposing spikes or teeth, or generally peaks and valleys and/or spaces between the prongs or teeth increase from the free ends of the clamping jaws to the closed ends of the clamp. As a result, at least approximately the same clamping force is possible, regardless of how far the auditory ossicle is arranged in the clamp from the free end along the longitudinal direction towards the closed end.

All of the features mentioned in the description and/or shown in the drawing can be implemented individually or in any combination in embodiments of the invention. Embodiments of the invention which do not have all but only some of the features of a claim, including those of the independent claim, are possible.

### DETAILED DESCRIPTION OF EMBODIMENTS

The invention is explained in more detail below with reference to an embodiment shown in the drawing. It is shown in:

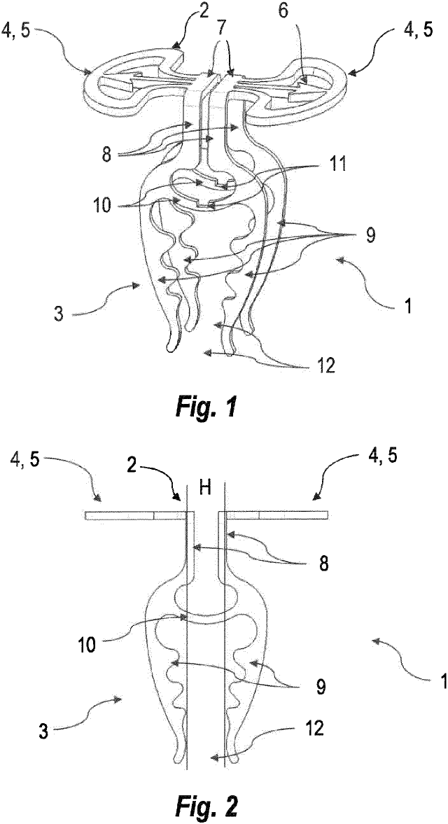

FIG. 1 a perspective view of an auditory ossicle prosthesis according to the invention;

FIG. 2 a side view of the auditory ossicle prosthesis from FIG. 1 in a initial position;

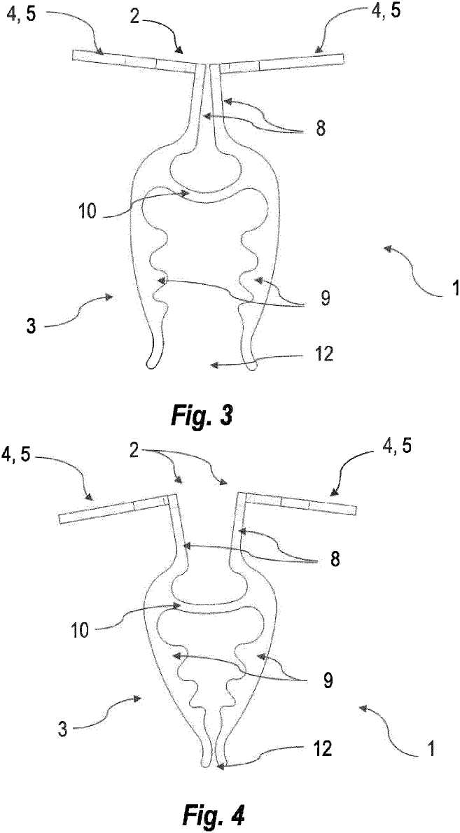

FIG. 3 the auditory ossicle prosthesis from FIG. 2 with an expanded clamp in a coupling position;

FIG. 4 the auditory ossicle prosthesis from FIG. 2 with narrowed clamp; and

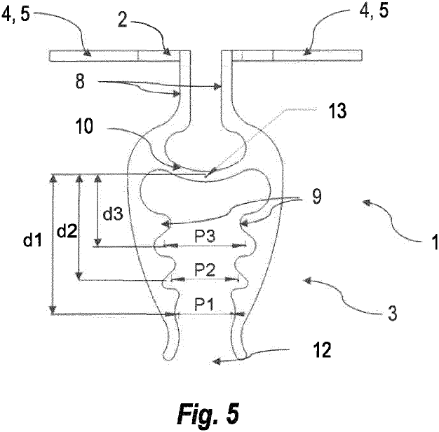

FIG. 5 the auditory ossicle prosthesis from FIG. 2 with dimensioned corrugation troughs.

The auditory ossicle prosthesis 1 according to the invention shown in the drawing is provided for replacing one or more auditory ossicles in a human middle ear. It has a head element ₂, which is intended to be arranged on an inside of an eardrum of the ear, and two clamps ₃, which are intended to be placed in a clamping manner on an auditory ossicle to fasten the auditory ossicle prosthesis 1 to the auditory ossicles. In particular, the clamps ₃ are placed on a leg of a stirrup in a person’s middle ear. However, they can also be placed on a long anvil leg of an anvil or a hammer handle of a hammer of the ear or attached at another point. The auditory ossicle prosthesis 1 transmits vibrations of the eardrum generated by sound waves to the stapes or the element of the ear to which the clamps ₃ are attached. In this context, an eardrum is understood to mean both an existing, partially existing and an artificially completely or partially reconstructed eardrum, as well as a partially existing and otherwise reconstructed eardrum. The reconstruction includes any form of production of a vibratory membrane, for example with body tissue or an artificially created vibratory membrane for converting sound into mechanical vibrations.

The head element ₂ has two head parts ₄ which can be moved relative to one another, namely whose distance from one another can be changed. In a view of the head element ₂, i.e. in a front view of the auditory ossicle prosthesis ₁, the head parts ₄ have Ω-shaped brackets ₅, which are arranged mirror-symmetrically to a central plane of the auditory ossicle prosthesis ₁, with “legs” of the Ω-shaped brackets ₅ facing each other. In their central plane, the Ω-shaped brackets ₅ have arrow-shaped bars 6 for stiffening purposes. The center plane of the auditory ossicle prosthesis 1 and the center plane of the Ω-shaped bracket ₅ are perpendicular to one another. The shape of the head parts ₄ is not mandatory for the invention; likewise, the head element ₂ does not have to be planar, but it can also be curved and/or voluminous (not shown).

The “legs” of the two Ω-shaped brackets ₅ of the head parts ₄ merge into bars ₇ in one piece, which run parallel to the center plane of the auditory ossicle prosthesis 1. At their ends, the two bars ₇ are bent at right angles or at least approximately at right angles to the same side of the head element ₂ into four shafts 8 which run parallel to the center plane of the auditory ossicle prosthesis ₁.

At their ends remote from the head element ₂, the shafts 8 merge-in one piece in the exemplary embodiment-into clamping jaws ₉, which extend arcuately away from the head element ₂ in a side view of the auditory ossicle prosthesis ₁. Convex outer sides or outer edges of the jaws ₉ face away from each other and concave inner sides or inner edges of the jaws ₉ face each other. Sections between the bars ₇ and the clamping jaws ₉ are referred to here as shafts 8.

The auditory ossicle prosthesis 1 has four clamping jaws ₉ which are associated with one another in pairs, with two associated clamping jaws ₉ each forming a clamp ₃ of the auditory ossicle prosthesis ₁. The two clamping jaws ₉, which are arranged mirror-symmetrically opposite one another with respect to the central plane of the auditory ossicle prosthesis ₁, are assigned to one another. The auditory ossicle prosthesis 1 has two clamps ₃ in two planes that are parallel to one another and perpendicular to the central plane of the auditory ossicle prosthesis ₁. In a side view, the terminals ₃ are congruent. It is also possible that the clamps ₃ are located in mutually inclined planes or curved surfaces and/or that the clamps ₃ are not congruent and/or that the clamping jaws ₉ are not mirror-symmetrical (not shown). The auditory ossicle prosthesis 1 preferably has two clamps ₃, although only one clamp ₃ or more than two clamps ₃ (not shown) are not excluded. The clamps ₃ can also be interpreted as U-shaped, because the legs of the “U’s” form the clamping jaws ₉.

The inner sides or inner edges of the clamping jaws ₉ facing one another have a corrugation, with the corrugations being of different heights in the exemplary embodiment: as the two parallel, straight auxiliary lines H in FIG. 2 and the dimensions in FIG. 5 illustrate, the distance from both the crests and the corrugations increases from troughs of corrugations of the insides or inner edges of the jaws ₉ from free ends of the jaws ₉, i.e. from an opening 13 of the clips ₃ towards closed ends of the clips ₃, i.e. towards the head element ₂. FIGS. 2 and 5 as well as FIG. 1 show the auditory ossicle prosthesis 1 in the preferred initial position, with the head parts ₄ being arranged planar. Other positions of the head parts ₄ in relation to one another are also conceivable, it being characteristic of the initial position that no pretension acts on the clamping jaws ₉ or head parts ₄ by means of elastically deformed bars ₁₀.

The clamping jaws ₉ are connected to one another in one piece by bars ₁₀ near their transitions into the shafts 8. In the exemplary embodiment, the bars ₁₀ are arced, which, however, is not essential for the invention. The bars ₁₀ connecting the clamping jaws ₉ are so narrow that they are deformable; in the exemplary embodiment, they are in particular bendable. The bars ₁₀ form a kind of joints which connect the clamping jaws ₉ pivotably with one another. If -- as shown in FIG. 3 -- the head parts ₄ are brought closer together, the clamping jaws ₉ swing apart and the clamps ₃ are opened. Conversely, if the head parts ₄ are removed from one another, the jaws ₉ pivot together and the clamps ₃ are closed, as shown in FIG. 4. This means that the clamps ₃ can be closed and opened like a pair of pliers, with the head parts ₄ acting as “pliers handles”. Conversely, as with many pliers, the clamps ₃ of the auditory ossicle prosthesis 1 are opened by bringing the head parts ₄ closer together. The invention does not exclude configurations of the auditory ossicle prosthesis 1 in which the clamps ₃ are closed by approaching the head parts ₄.

Deviating from FIGS. 2 to 4, the bars ₁₀ in FIG. 1 have a narrow point 11 which makes it easier for the bars ₁₀ to be deformable. The bars ₁₀ are the only connections of the clamping jaws ₉ of the clamps ₃ and, via the shafts 8, also of the head parts ₄ with the Ω-shaped brackets ₅ of the head element ₂ of the auditory ossicle prosthesis 1 according to the invention. Otherwise, the head parts ₄ and the clamping jaws ₉ are separate from one another; they are arranged mirror-symmetrically about the center plane of the auditory ossicle prosthesis 1 on both sides of the center plane at a distance from one another.

The bars ₇ on the “leg” of the Ω-shaped bracket ₅ and the shafts 8 are rigid or at least more rigid than the bars ₁₀ which connect the jaws ₉ pivotably as joints. The shafts 8 and the bars ₇ are in any case so stiff that they can be moved apart or together by moving the head parts ₄ together or apart, the jaws ₉ and thus the clamps ₃ can be opened and closed, even if the bars ₇ and the Shafts 8 deform slightly elastically.

In order to attach the auditory ossicle prosthesis 1 according to the invention to an auditory ossicle, for example to a stirrup in a middle ear and there in particular to a leg of the stirrup without exerting any force other than a clamping force on the auditory ossicle, the clamps ₃ of the auditory ossicle prosthesis 1 are opened, i.e. the clamping jaws ₉ pivoted apart by the head parts ₄ are moved towards each other, as FIG. 3 shows. For this purpose, a surgeon can grip the auditory ossicle prosthesis 1 from a side of the head element ₂ opposite the clamps ₃ using, for example, forceps at the head parts ₄ and press the head parts ₄ together. The clamps ₃ thus opened can be placed on the auditory ossicle without exerting a force on the auditory ossicle. If the head parts ₄ are now released, a spring force of the bars ₁₀, which are elastically deformed when the clamps ₃ are opened, closes the clamps ₃ and presses the clamping jaws ₉ against the ossicle from two opposite sides, so that the ossicular prosthesis 1 is clamped to the ossicle.

In order to adapt to auditory ossicles of different thicknesses, the auditory ossicle prosthesis 1 can be plastically deformed before it is placed on the auditory ossicles. In particular, the bars ₁₀ connecting the clamping jaws ₉ are plastically deformed. FIG. 4 shows, for example, a plastic bending together of the clamping jaws ₉ in order to adapt the clamps ₃ to a thinner auditory ossicle, for example to a leg of a stirrup with a small diameter.

By placing the clamps ₃ on the ossicles at different distances, so that the ossicles are in a corrugation trough of the clamping jaws ₉, a distance between the head element ₂ and the ossicle can be set and adapted to a distance between the eardrum and the ossicle.

The different distances between the crests and troughs of the corrugations on the insides or inner edges of the clamping jaws ₉ ensure that the clamping forces of the clamping jaws ₉ on the ossicles are not too high when the ossicles are clamped close to the closed end of the clamps ₃, where due to the short distances from or the short lever arms to the bars ₁₀ act higher forces than near the open end of the clamps ₃, where the distances from the bars ₁₀ or the lever arms are longer.

This advantageous embodiment of the invention is shown in FIG. 5 by the dimensioning of the auditory ossicle prosthesis 1 in its initial position. d₁, d₂ and d₃ designate the respective distance of the corrugation troughs from the closed end of the clamps ₃, measured from the bars ₁₀. The respective distances between opposite troughs of the corrugations of the inner sides or inner edges of the clamping jaws ₉ are denoted by P₁, P₂ and P₃. The distance is largest in the trough at P₃, the smallest in the trough at P₁ and the distance in the trough at P₂ is less than at P₃ but larger than at P₁. This applies in the same way to the distances between opposing corrugation crests, the distances of which increase towards the closed end of the clamps ₃.

In a further advantageous embodiment of the auditory ossicle prosthesis 1 according to the invention, the products formed from the distances between opposite troughs and the respective distance of these troughs from the pivot point 13 predetermined by the bar ₁₀ at the closed end are the same. As a result, the holding force that acts on the ossicles as a result of the preloading is always the same, regardless of the trough in which the ossicles are fixed in the longitudinal direction on the clamping jaws ₉. Illustrated using the example of FIG. 5, this corresponds to the following relationship: P₁ · d₁ = P₂ · d₂ = P₃ · d₃, the distances d₁, d₂ and d₃ being measured from a pivot point 13 specified by the bar ₁₀ around which the clamping jaws ₉ rotate elastic deformation of the bar ₁₀ pivot. As stated above, this embodiment of the invention is in no way limited to mutually facing inner surfaces or inner edges of the clamping jaws ₉ which have a corrugation. Inner surfaces or inner edges of the clamping jaws ₉ designed as prongs or teeth are also suitable. In fact, any shape of the inner surfaces or inner edges of the jaws ₉ having valleys and peaks is suitable.

In a further advantageous embodiment, the auditory ossicular prosthesis 1 according to the invention is designed in such a way that, in the initial position of the auditory ossicular prosthesis ₁, a distance between opposite peaks on the inner surfaces or inner edges of the clamping jaws ₉ increases from a free end to a closed end of the clamp ₃. Additionally or alternatively, the auditory ossicle prosthesis 1 according to the invention can be designed in such a way that in the coupling position of the auditory ossicle prosthesis 1 a distance between opposite peaks from one another decreases from a free end to a closed end of the clamp ₃. This has the further advantage that when the auditory ossicle prosthesis 1 is in the coupling position for application to an auditory ossicle, the auditory ossicle is not damaged when it is guided from the free end to the closed end of the clamp ₃ along the longitudinal direction of the clamping jaws ₉ for application, because it is does not roam past peaks, regardless of how far or where the auditory ossicles are fixed in the longitudinal direction on the clamping jaws ₉ in the clamp ₃.

The auditory ossicle prosthesis 1 according to the invention is made in one piece, it is separated from sheet metal by laser cutting and bent to form the auditory ossicle prosthesis ₁. Other types of production and/or production from several parts are possible.

1. Auditory ossicle prosthesis comprising:
a head element, which is provided for an arrangement on an eardrum; and
a clamp having two clamping jaws configured to be clamped to an auditory ossicle, so that the auditory ossicle is between the two clamping jaws and the auditory ossicle prosthesis is clamped to the auditory ossicle, wherein the head element has several head parts configured to be moved relative to one another, and one head part is connected to a one clamping jaw and another head part (4)-is connected to another clamping jaw in such a way that the two clamping jaws (9)-can be moved away from one another and/or brought closer to one another by moving the head parts relative to one another.

2. Auditory ossicle prosthesis according to claim 1, wherein the clamping jaws are configured to be moved apart by moving the head parts apart from one another and/or by moving the head parts (4)-closer together, or the clamping jaws are configured to be moved closer together by moving the head parts closer together and/or by moving the head parts apart from one another.

3. Auditory ossicle prosthesis according to claim 1, further comprising a deformable bar configured to connects the clamping jaws (9)-at an adjustable distance.

4. Auditory ossicle prosthesis according to claim 1, wherein the auditory ossicle prosthesis is designed in the manner of resilient pliers, with the head parts (4)-forming pliers handles or parts of pliers handles which, when moving away from one another or approaching the clamping jaws move towards or away from one another and/or that the clamping jaws (9)-spring back elastically into an initial position.

5. Auditory ossicle prosthesis according to claim 1, wherein a distance between the clamping jaws is configured to be adjusted by plastic deformation and/or that the clamp (3)-is elastically deformable, so that a distance between the clamping jaws (9)-can be an elastic deformation of the clamp can be changed.

6. Auditory ossicles prosthesis according to claim 1, wherein the clamp is U-shaped, the legs of the “U” forming the clamping jaws.

7. Auditory ossicle prosthesis according to claim 1, wherein the auditory ossicle prosthesis has two or more congruent clamps configured to be clamped onto the auditory ossicle.

8. Auditory ossicle prosthesis according to claim 1, wherein mutually facing inner surfaces or inner edges of the clamping jaws (9)-have a corrugation, spikes or teeth each with at least one valley and at least one peak.

9. Auditory ossicle prosthesis according to claim 8, wherein a distance between opposite valleys increases from a free end of the clamping jaws (9)-to a closed end of the clamp.

10. Auditory ossicle prosthesis according to claim 9, wherein the products formed from the distances between opposite valleys and the respective distance of these valleys from a pivot point specified by the bar are the same at the closed end.

11. Auditory ossicle prosthesis according to claim 8, wherein in an initial position of the auditory ossicle prosthesis, a distance between opposite peaks increases from a free end to a closed end of the clamp.

12. Auditory ossicle prosthesis according to claim 8, wherein in a coupling position of the auditory ossicle prosthesis, a distance between opposing peaks decreases from a free end to a closed end.

13. Auditory ossicle prosthesis according to claim 1, wherein the auditory ossicle prosthesis is in one piece.
